(19) United States
(12) Patent Application Publication
(10) Pub. No.: US 2017/0049423 A1
(43) Pub. Date: Feb. 23, 2017

(54) DEVICE FOR IN VIVO SAMPLING OF BIOLOGICAL SPECIES

(71) Applicant: COMMISSARIAT A L'ENERGIE ATOMIQUE ET AUX ENERGIES ALTERNATIVES, Paris (FR)

(72) Inventors: Adrien Mombrun, Grenoble (FR); Mohamed- Ali Bouamrani, Grenoble (FR); Eric Boyer, Meylan (FR); Christelle Yeromonahos, Lyon (FR)

(73) Assignee: COMMISSARIAT A L'ENERGIE ATOMIQUE ET AUX ENERGIES ALTERNATIVES, Paris (FR)

(21) Appl. No.: 15/307,447

(22) PCT Filed: Apr. 30, 2015
(86) PCT No.: PCT/EP2015/059450

§ 371 Date: Oct. 28, 2016

(30) Foreign Application Priority Data
Apr. 30, 2014 (FR) 1453945

(51) Int. Cl. A61B 10/02; A61B 10/00

(52) U.S. Cl. CPC A61B 10/0233; A61B 10/0045; A61B 2010/0208; A61B 2010/0054

(57) ABSTRACT

The invention relates to a device for in vivo sampling of biological species, comprising:
a tubular sheath extending between a proximal end of said sheath and a distal end of said sheath, said distal end of the sheath having a projecting part,
a rod extending between a proximal end of said rod and a distal end of said rod, capable of sliding in the sheath between a retracted position in which the distal end of the rod is located inside the sheath and a deployed position in which the distal end of the rod extends beyond the distal end of the sheath,
   said rod comprising a capturing support for capturing said biological species, made from a porous material, arranged in a distal region of the rod on a portion of the circumference of the rod such that the capturing support is located outside the sheath when the rod is in the deployed position of same.

### FIELD OF THE INVENTION

The present invention relates to a device for in vivo sampling of biological species.

### BACKGROUND OF THE INVENTION

Cellular and molecular mechanisms have been highlighted in the alteration of the auditory function, notably in presbycusis and acoustic trauma.

It seems in particular that proteins contained in the perilymph affect the metabolism and the distribution of medicines delivered to the cochlea. Numerous proteins seem in fact to attach themselves to the released medicines, rendering them inactive against the targeted tissues [1].

An analysis of the perilymph has been carried out on patients presenting vestibular schwannoma, providing an insight into the proteins contained in the perilymph [2]. This study highlighted the presence of two proteins in the perilymph of patients suffering from vestibular schwannoma. Its authors lay stress on the interest of a collection of the perilymph without loss of material for the analysis of the proteome and report the existence of contamination of the collected sample by elements of the blood (haemoglobin, keratin).

In the literature, analyses of the perilymph obtained by external samplings have been described. However, these samplings are subject to contamination by cerebrospinal fluid or resort to very complex methods.

Thus, a study conducted on guinea-pigs shows that the procedure for sucking up samples at the level of the basal portion of the cochlea of the scala tympani has an important influence on the composition of the perilymph [3]. Although the part sucked up initially seems to contain pure perilymph, the sample is contaminated with the sucking up of cerebrospinal fluid. The samplings made at the level of the basal portion of the cochlea are thus in reality a mixture of the perilymph and cerebrospinal fluid.

The authors of the study [3] have developed a novel method for obtaining larger samples of the perilymph while collecting it without any loss at the level of the cochlear apex [4]. Nevertheless, sampling biases made at the level of the basal portion of the cochlea remain a major source of error in the understanding of the pharmacokinetics of the perilymph. Furthermore, the sampling protocol according to this method is very ponderous and cannot be carried out in practice in vivo.

A standardised sampling methodology is thus necessary for collecting a body fluid present in a cavity, in particular the perilymph when the cavity is the round window of the cochlea.

### BRIEF DESCRIPTION OF THE INVENTION

An aim of the invention is to design a device for sampling biological species in the cochlea or other glands of small dimensions, which minimises or even avoids any contamination of the biological species during sampling.

In accordance with the invention, a device is proposed for in vivo sampling of biological species comprising:
a tubular sheath extending between a proximal end of said sheath and a distal end of said sheath, said distal end of the sheath having a projecting part suited to perforating a membrane of an organ containing the biological species to sample,
a rod extending between a proximal end of said rod and a distal end of said rod, capable of sliding in the sheath between a retracted position in which the distal end of the rod is located inside the sheath and a deployed position in which the distal end of the rod extends beyond the distal end of the sheath,
   said rod comprising a capturing support for capturing said biological species, made from a porous material, arranged in a distal region of the rod on a portion of the circumference of the rod such that the capturing support is located outside the sheath when the rod is in the deployed position of same.

The terms “proximal” and “distal” are defined herein with respect to the practitioner who handles the device.

“Porous” is herein taken to mean a pore density comprised between 10 and 75% and a pore size comprised between 1 and 100 nm.

Biological species is taken to mean proteins, peptides, metabolites, or any organic molecule capable of being present in an organism. It may also be cells, bacteria, viruses, or other microorganisms.

According to an embodiment, the distal end of the tubular sheath forms a bevel.

In a particularly advantageous manner, the distal end of the rod comprises a rounded tip.

According to an embodiment, said tip is made from a biocompatible polymer.

To this end, the rod has a housing for the capturing support, said housing being arranged such that the surface of the capturing support is set back from the circumferential surface of the rod.

In a particularly advantageous manner, the capturing support comprises nanoporous silicon or an organosilicon material.

According to an embodiment, the distal region of the rod comprising the capturing support is breakable.

The sheath may advantageously be made from polytetrafluoroethylene.

According to a form of execution, the proximal end of the rod is coupled to an actuating means.

### BRIEF DESCRIPTION OF THE DRAWINGS

Other characteristics and advantages of the invention will become clear from the detailed description that follows, with reference to the appended drawings among which:

FIG. 1 is a side view of a sampling device in accordance with the invention, in the retracted configuration of same,

FIG. 2 is a side view of a sampling device in accordance with the invention, in the deployed configuration of same,

FIG. 3 shows MALDI analysis spectra of a sample of the perilymph on a conventional stainless steel surface (a) and on the surface of a capturing support used in the invention (b).

### DETAILED DESCRIPTION OF THE INVENTION

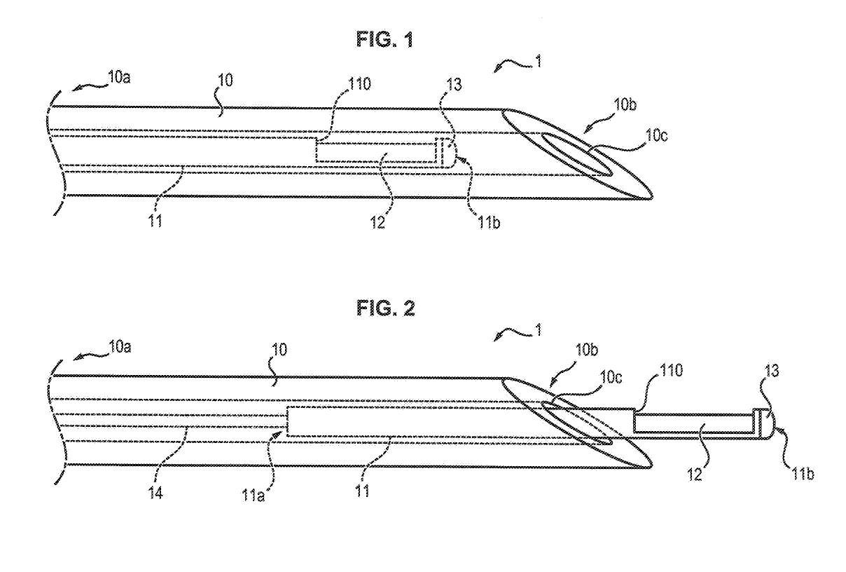

FIG. 1 illustrates a device for in vivo sampling of biological species in the retracted configuration of same. FIG. 2 illustrates said device in the deployed configuration of same.

The device 1 comprises a tubular sheath 10 extending between a proximal end 10a and a distal end 10b.

The distal end 10b has a projecting part extending parallel to the longitudinal axis of the sheath, suited to perforating the membrane surrounding the organ in which the sampling must be carried out.

For example, said distal end has a bevelled shape. The bevel angle is sufficiently pronounced to enable an incision of the membrane without a high load pressure being applied to the organ. This bevel may be produced by a machining of the distal end of the tubular sheath.

Advantageously, said distal end is sharp, in order to facilitate the incision and minimise damage to the tissues passed through.

The tubular sheath 10 has a cylindrical interior channel emerging at the distal end 10b via an opening 10c.

The device 1 further comprises a rod 11 extending between a proximal end 11a and a distal end 11b.

The rod 11 is capable of sliding in the inner channel of the sheath 10 between a retracted position in which the distal end 11b of the rod is located inside the sheath 10 (cf. FIG. 1) and a deployed position in which the distal end 11b of the rod 11 extends beyond the distal end 10b of the sheath, through the opening 10c (cf. FIG. 2).

The rod 11 comprises a capturing support 12 for capturing said biological species, which is made from a porous material.

Advantageously, the rod 11 is made from a biocompatible material, such as poly ether ether ketone (PEEK).

Advantageously, the support 12 is made from nanoporous silicon or an organosilicon material and made integral with the rod 11 by means of a biocompatible adhesive or any other appropriate means of fixation.

To this end, the rod advantageously has in a portion of its circumference a housing 110 of which the dimensions are suited to receiving the support 12. The support 12 is advantageously arranged set back in the housing 110 with respect to the circumferential surface of the rod, such that its edges, which may be sharp-edged, do not aggress the organ in which the sampling is carried out. Furthermore, this arrangement also avoids any friction between the capturing support 12 and the inner wall of the sheath 10 during the handling of the rod 11.

The capturing support 12 is arranged in a distal region of the rod 11, such that said support 12 is located outside the sheath 10 when the rod 11 is in the deployed position of same (cf. FIG. 2).

Advantageously, the distal part of the rod including the capturing support is breakable, which makes it possible to have available easily the capturing support with a view to the analysis of the sampled species.

At its proximal end 11a, the rod 11 is coupled to an actuating means 14. Said means extend in the proximal direction beyond the proximal end of the sheath 10 and are intended to be handled by the practitioner to make the rod slide between the retracted position and the deployed position. Said actuating means 14 advantageously comprise a cable or a flexible tube, having a smaller diameter than that of the rod 11 and having sufficient rigidity to exert a pushing of the rod.

At its distal end 11b, the rod 11 preferably has a rounded tip 13 so as not to cause any lesion of the organ in which the sampling is carried out. Said tip 13 may form an integral part of the rod and thus be formed of the same material as said rod, or instead be made from another material then made integral with the rod by any appropriate means.

The sheath is advantageously made from polytetrafluoroethylene (PTFE) or another material having a low coefficient of friction.

Thus, the sheath protects the rod and the capturing support located in the sheath before and after the actual sampling, while facilitating the sliding of the rod between the retracted position and the deployed position of same.

The capturing support is only exposed to the tissues or to the body fluids of the patient when the distal end of the sheath 10 has been placed at the suitable spot in the organ in which the sampling must be carried out.

Purely as an indication, the sheath 10 has a length of at least 10 cm, an external diameter comprised between 1 and 2 mm and an internal diameter comprised between 0.7 and 1.3 mm.

The rod 11 has a diameter comprised between 0.3 and 0.7 mm and a length of at least 2 cm.

As indicated above, the capturing support 12 preferably has a shape inscribed within the diameter of the rod 11. The length of the support 12 can vary as a function of the sampling conditions provided and is typically comprised between 1 mm and 1 cm.

The capturing support is advantageously made from nanoporous silicon, experience having shown that this material is well suited to the capture of small proteins, that is to say of which the m/z ratio (where m designates the mass of the molecule and z the charge) is less than 8000 or even 1000.

The nanoporosity of the surface of the capturing support may be obtained from different materials. Depending on the method employed, the porous thickness, the porosity density and the size of the pores can vary. The variation in these characteristics modifies the nature of the molecules (depending on the weight range) captured and analysable by MALDI mass spectrometry. Typically, the porous thickness is greater than 100 nm, the pore density is comprised between 10 and 75% and the pore size is comprised between 1 and 100 nm.

This nanoporous surface may result from an electrochemical attack of a silicon substrate. The thickness over which the substrate is made porous is typically greater than 100 nm and may be the entire thickness of the substrate. For example, it is possible to obtain by electrochemical attack a porous thickness of the order of 2.2 μm, a porosity density of 40% and a size comprised between 10 and 15 nm.

Alternatively, the nanoporous surface may result from the deposition of a layer of a porous organosilicon material, of SiOCH type, on a substrate. For example, a SiOCH layer is deposited by PECVD (acronym for the term Plasma Enhanced Chemical Vapour Deposition) by joint deposition of an organosilicon matrix and thermally labile organic compounds (pore-forming agents). The pore forming agents are then evacuated by a UV annealing at 400° C. for several minutes. The SiOCH layer thereby obtained has a controlled thickness that is able to be comprised between 180 nm and 1000 nm, an open porosity and interconnected maximum of 30% (ellipsoporosimetry measurement using toluene), a mean diameter of the pores of 1.3 nm and is hydrophobic with a contact angle of the order of 100°. The physical-chemical characteristics of the layer can then be modified by plasma post-treatment. Thus, an N2H2 plasma makes it possible to obtain a porosity of 35% and a contact angle of 80°.

Alternatively, the capturing support may be made from metal rendered nanoporous by electrochemical attack (for example based on hydrofluoric acid), or comprise a layer of porous organic polymer (for example di-block copolymer) deposited on a substrate.

The protocol for introducing the device 1 for the sampling of the perilymph in the cochlea is similar to that of putting in place a cochlear implant.

The device is introduced in the following manner: the approach up to the cochlea is carried out firstly by retroauricular route with carrying out of a masto-antro-atticotomy then carrying out of a posterior tympanotomy. The round window membrane is then exposed. The device may then be introduced. During this step of introduction, the device is in the retracted position of same, the capturing support thus not being exposed to tissues and biological fluids.

The gesture of the practitioner is guided optically (binocular), such that the practitioner visualises the position of the projecting part of the distal end of the sheath with respect to the membrane.

Once the membrane is pierced, the practitioner ceases the introduction of the sheath and deploys the rod so as to place the capturing support in contact with the perilymph. In this way are sampled on the capturing support biological species capable of being involved in hearing malfunctions, such as proteins (notably lipofuscin).

During this operation, the tip 13 of the rod may potentially come to press on the bony shell inside the cochlea. The fact that this tip is rounded makes it possible to avoid causing a lesion of the cochlea.

Then the rod is again reinserted into the tubular sheath and the retracted device is removed from the body of the patient.

The example described above is only a particular illustration which is not limiting with regard to the application fields of the invention. Thus, apart from the sampling of the perilymph in the cochlea, the device described above may also be used for samplings of biological fluids in glands such as the salivary glands, the lacrimal glands or instead the sinuses.

### Experimental Example

To validate the capacity of the capturing support made from nanoporous silicon to sample biological species, the protocol described hereafter was implemented from a sampling of the perilymph during an operation on a rat.

The sampling volume was ten or so microlitres.

Two 5 mm by 5 mm silicon chips with pores of 1 to 2 nm (SiOCH layer deposited by PECVD as described above), cleaned by ultra-sonification in acetone for 3 min, were prepared.

9AA (9-aminoacridine) suitable for MALDI analysis of metabolites and peptides (up to 1000 Daltons) was used on the SiOCH surface which, by virtue of the dimension of its pores, essentially enriches this type of molecule.

1 μL of perilymph was deposited on one of the two silicon chips, the other being left bare as a control.

9M (9-aminoacridine—MALDI matrix) was deposited on the two chips, in order to study the metabolome of the perilymph.

The perilymph was incubated for 5 minutes on the porous support before rinsing with an aqueous 0.1% TFA solution and analysed by MALDI mass spectrometry.

In order to validate the interest of nanoporous SiOCH, the same sample of the perilymph was analysed in a conventional manner on a MALDI plate having a non-porous stainless steel surface.

Reading parameters on the MALDI mass spectrometer (Brucker Ultraflex): the automatic acquisition of the spectra was realised on 5000 laser impacts in positive reflectron mode, a laser intensity at 75% and an attenuation of the matrix signal at 200 Daltons and at 0 Daltons. in other words, molecules of which the m/z ratio is less than 200 Daltons, mainly stemming from the 9-aminoacridine matrix, are deviated in order to avoid saturation of the detector.

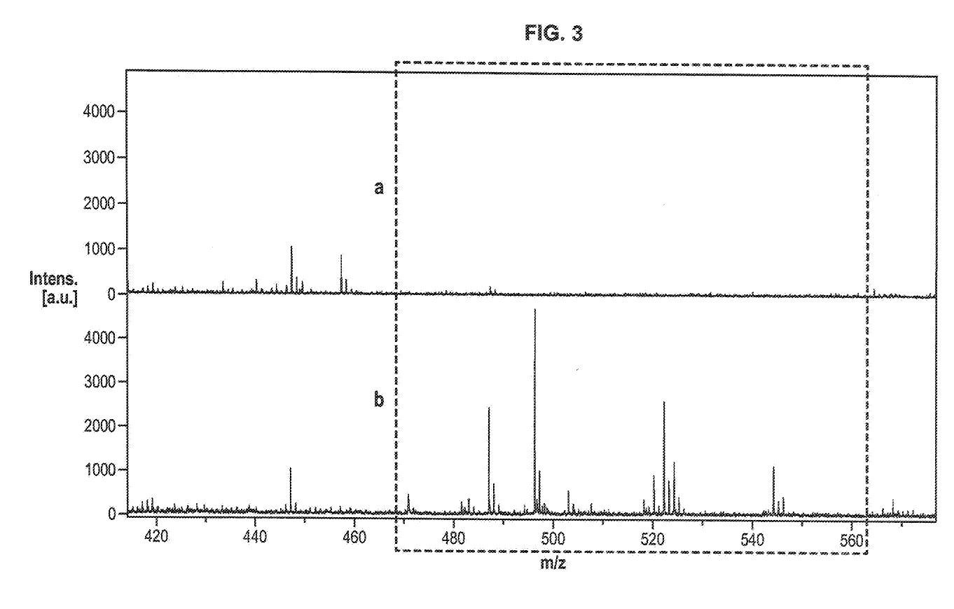

FIG. 3 shows in the upper part (a) the spectrum of the perilymph on the stainless steel surface and in the lower part (b) the spectrum of the perilymph on the surface of the porous silicon. The x-axis is graduated according to a mass scale (m/z); the y-axis corresponds to an arbitrary scale, identical for both spectra.

In the surrounded area may be observed a visible enrichment of the protein, peptide and metabolomic spectrum on the porous silicon surface to compared to the stainless steel surface.

### REFERENCES

[1] Swan E E, Peppi M, Chen Z, Green K M, Evans J E, McKenna M J, Mescher M J, Kujawa S G, Sewell W F. Proteomics analysis of perilymph and cerebrospinal fluid in mouse. Laryngoscope. 2009 May; 119(5):953-8

[2] Lysaght A C, Kao S Y, Paulo J A, Merchant S N, Steen H, Stankovic K M. Proteome of human perilymph. J Proteome Res. 2011 Sep. 2; 10(9):3845-51

[3] Salt A N, Kellner C, Hale S. Contamination of perilymph sampled from the basal cochlear turn with cerebrospinal fluid. Hear Res 2003; 182:24-33

[4] Salt A N, Hale S A, Plontke S K. Perilymph sampling from the cochlear apex: a reliable method to obtain higher purity perilymph samples from the scala tympani. J Neurosci Meth 2006; 153:121-129

1. A device for in vivo sampling of biological species, comprising:
a tubular sheath extending between a proximal end of said sheath and a distal end of said sheath, said distal end of the sheath having a projecting part adapted to perforate a membrane of an organ containing the biological species to sample.
a rod extending between a proximal end of said rod and a distal end of said rod, capable of sliding in the sheath between a retracted position in which the distal end of the rod is located inside the sheath and a deployed position in which the distal end of the rod extends beyond the distal end of the sheath,
wherein said rod comprises a capturing support for capturing said biological species, made from a porous material, arranged in a distal region of the rod on a portion of a circumference of the rod such that the capturing support is located outside the sheath when the rod is in the deployed position of same.

2. The device of claim 1, wherein the distal end of the tubular sheath forms a bevel.

3. The device of claim 1, wherein the distal end of the rod comprises a rounded tip.

4. The device of claim 3, wherein said tip is made from a biocompatible polymer.

5. The device of claim 1, wherein the rod has a housing for the capturing support, said housing being arranged such that the surface of the capturing support is set back from the circumferential surface of the rod.

6. The device of claim 1, wherein the capturing support comprises nanoporous silicon or an organosilicon material.

7. The device of claim 1, wherein the distal region of the rod comprising the capturing support is breakable.

8. The device of claim 1, wherein the sheath is made from polytetrafluoroethylene.

9. The device of claim 1, wherein the proximal end of the rod is coupled to an actuating means.
